(19) United States
(12) Patent Application Publication
(10) Pub. No.: US 2026/0014106 A1
(43) Pub. Date: Jan. 15, 2026

(54) COMBINATION IMMUNORESPONSE REGULATOR/IMMUNOTHERAPY SYSTEM AND METHOD

(71) Applicant: KODISCOVERY, LLC, Baltimore, MD (US)

(72) Inventor: Young Hee KO, Baltimore, MD (US)

(21) Appl. No.: 18/992,425

(22) PCT Filed: Jul. 10, 2023
(86) PCT No.: PCT/US2023/069904

§ 371 Date: Jan. 8, 2025

Related U.S. Application Data
(60) Provisional application No. 63/359,674, filed on Jul. 8, 2022.

(51) Int. Cl. A61K 31/19; A61K 31/137; A61K 31/7004; A61K 47/12; A61K 47/26

(52) U.S. Cl. CPC A61K 31/19; A61K 31/137; A61K 31/7004; A61K 47/12; A61K 47/26

(57) ABSTRACT

A system for the treatment of a condition susceptible to overactivation of a subject's immune system, is described and discussed. The system includes an immunotherapeutic capable of treating a condition by modulating activity of a subject's immune system and an immunoresponse regulator composition that includes 3-bromopyruvic acid (3-BP) and salts thereof, at least one sugar to stabilize the 3-BP by substantially preventing the 3-BP from hydrolyzing, and a biological buffer present in an amount sufficient to at least partially deacidify and neutralize metabolic by-products of the 3-BP.

### CROSS-REFERENCE TO RELATED APPLICATIONS

This application claims the benefit of U.S. Provisional Patent Application No. 63/359,674, filed on Jul. 8, 2022, which is incorporated herein by reference in its entirety.

### SEQUENCE LISTING

This application contains a sequence listing which is incorporated herein by reference in ST.26 XML format named 2553-052.PCT SEQ Listing.xml, created Jul. 10, 2023, and is 14 KB in size. The sequences contained in the sequence listing are found throughout the originally filed application.

### BACKGROUND

The immune system is a large collection of organs, specialized cells, and substances that help protect the body from infections, pathogens, and various other diseases. The immune system tracks the various substances normally found in the body to avoid triggering specialized immune cells immune cells that would otherwise target the body's tissue. Any substance that the immune system doesn't recognize, however, generates a signal that causes the immune system to attack it. For example, pathogens have surface proteins that are not recognized by the immune system proteins found in the human body. The immune system sees these as “foreign” and activated immune cells attack the pathogens, thus generating an immune response that can destroy the foreign substance and anything associated with it.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only, and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 0.01%. It is to be understood that, even when the term “about” is used in the present specification in connection with a specific numerical value, support for the exact numerical value recited apart from the “about” terminology is also provided.

As used herein, a plurality of ingredients, conditions, and/or compositional components may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The formulations of the present invention may include a pharmaceutically acceptable carrier and other ingredients as dictated by the particular needs of the specific dosage formulation. Such ingredients are well known to those skilled in the art. See for example, Gennaro, A. Remington: The Science and Practice of Pharmacy 19th ed. (1995), which is incorporated by reference in its entirety.

As used herein, “administration,” and “administering” refer to the manner in which a composition is presented to a subject. Administration can be accomplished by various art-known routes such as enteral, parenteral, transdermal, and the like, including combinations thereof in some cases. Thus, an enteral administration can be achieved by drinking, swallowing, chewing, sucking of an oral dosage form comprising an active agent or other compound to be delivered. Parenteral administration can be achieved by injecting a drug composition intravenously, intra-arterially, intramuscularly, intrathecally, subcutaneously, etc. Transdermal administration can be accomplished by applying, pasting, rolling, attaching, pouring, pressing, rubbing, etc., of a transdermal preparation onto a skin surface. These and additional methods of administration are well-known in the art.

As used herein, the terms “subject” and “subject” can be used interchangeably when the context allows, and refer to a mammal that may benefit from the administration of a drug composition or method of this invention. Examples of subjects include humans, and other animals such as horses, pigs, cattle, sheep, goats, dogs (felines), cats (canines), rabbits, rodents, primates, and aquatic mammals. In one embodiment, the subject can refer to a human.

As used herein, “cellular energy inhibitor” refers to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation is intended to include the active agent per se, as well as pharmaceutically acceptable salts, or compounds significantly related thereto, including without limitation, prodrugs, active metabolites, isomers, and the like. The terms “cellular energy inhibitor,” “glycolysis inhibitor,” “mitochondrial inhibitor,” and the like, are considered to be active agents.

As used herein, the terms “inhibit,” “inhibiting,” or any other derivative thereof refers to the process of holding back, suppressing or restraining so as to block, prevent, limit, or decrease a rate of action or function. The use of the term is not to be misconstrued to be only of absolute prevention but can be a referent to any minute incremental step of limiting or reducing a function through the full and absolute prevention of the function.

As used herein, “cellular energy inhibitor” refers to a compound that inhibits ATP production in a cell. In some examples, a cellular energy inhibitor can inhibit glycolysis, oxidative phosphorylation, or both glycolysis and oxidative phosphorylation in a cell.

As used herein, “glycolysis inhibitor” refers to a compound that inhibits, reduces, or stops, glycolysis in a cell.

As used herein, “mitochondria inhibitor” refers to a compound that inhibits, reduces, or stops mitochondrial production of ATP in a cell.

As used herein, “carrier” or “pharmaceutically acceptable carrier” refers to a substance with which a drug may be combined to achieve a specific dosage formulation for delivery to a subject. In some examples, a carrier may or may not enhance drug delivery. As a general principle, carriers do not react with the drug in a manner that substantially degrades or otherwise adversely affects the drug, except that some carriers may react with a drug to prevent it from exerting a therapeutic effect until the drug is released from the carrier. Further, the carrier, or at least a portion thereof must be physiologically suitable for administration into a subject along with the drug.

The term “excipient” herein includes any substance used, for example, as a carrier for an active agent in a liquid formulation, any substance added to the active agent and/or a solid formulation to, for example, improve its handling properties, permit the resulting composition to be formed into an appropriate storage form, facilitating disintegration in a liquid, or the like. Excipients can include, by way of illustration and not by limitation, diluents, disintegrants, binding agents, adhesives, wetting agents, lubricants, glidants, dyes, and any other substance other than the active ingredient conventionally used in the preparation of a liquid or solid formulation.

The terms “reaction” and “react” include any form of chemical change that occurs to a formulation ingredient as a result of contact with another formulation ingredient, including reactions that activate one or more molecules or ingredients (e.g., the change of a precursor to an active agent into the active agent), reactions that degrade at least one ingredient, or the like.

As used herein, “admixed” means that at least two components of the composition can be partially or fully mixed, dispersed, suspended, dissolved, or emulsified in one another. In some cases, at least a portion of the drug may be admixed in at least one carrier substance.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

### DETAILED DESCRIPTION

The immune system is a large collection of organs, specialized cells, and substances that help protect the body from infections, pathogens, and various other diseases. The immune system tracks the various substances normally found in the body to avoid triggering specialized immune cells immune cells that would otherwise target the body's tissue. Any substance that the immune system doesn't recognize, however, generates a signal that causes the immune system to attack it. For example, pathogens have surface proteins that are not recognized by the immune system proteins found in the human body. The immune system sees these as “foreign” and activated immune cells attack the pathogens, thus generating an immune response that can destroy the foreign substance and anything associated with it.

The immune system can have some difficulty, however, targeting cancer cells because cancer is an aberration of normal cells that the immune system doesn't recognize as foreign. This lack of recognition, or diminished recognition, hampers the immune system's ability to fight cancer on its own. Immunotherapy is one very general category of treatments that utilize the body's own immune system to combat cancer. But stimulating a subject's immune system to combat cancer can be risky, often leading to serious conditions, such as autoimmune disorders, tissue damage, and even death. In such cases, the immune system becomes hyperactivated and begins to attack normal cells and tissues in the body.

One mechanism that plays a key role in a hyperactivated immune system and the subsequent damage caused relates to the communication pathway immune cells use to coordinate their response to foreign material. The cells involved in the body's immune response coordinate their attack by releasing proteins that serve as chemical messengers. Cytokines are one type of protein used as a chemical message and thus are an integral part of the body's immune response. Cytokines are often crucial to various immunotherapy treatments against cancer, as well as other pathogenic attacks, diseases, and conditions. Cytokines can trigger fever, inflammation, runny nose, and body aches often associated with the flu, for example.

But in immunotherapy treatments, cytokine production can very easily grow out of control. Basically, immune cells release cytokines that tell the immune system to produce more immune cells, which results in these new cells releasing even more pro-inflammatory cytokines. A subset of cytokines, known as chemokines, are critical in the recruitment of cells to sites of inflammation—and help to fight pathogens—but this process can have a detrimental overall effect. Cytokine production can enter a positive feedback loop, thus leading to a so called “cytokine storm,” a situation in which excessive cytokine production causes an immune response that triggers an increase in cytokine production which can damage organs, especially the lungs and kidneys, and even lead to death. As such, the stimulation of the body's immune system to combat cancer can cause hyperactivation of the cytokine signaling pathway, which, at least in the short term, can be far more dangerous than the cancer itself.

In one example, the present disclosure provides systems and methods for treating a cancer or other condition with a combination of immunotherapy to increase the activity of the immune system and an immunoresponse regulator (i.e., energy inhibitor). In some examples, the immunoresponse regulator can function to eliminate or otherwise pacify hyperactivated immune cells. Immunoresponse regulators function to inhibit energy production within immune cells that have been hyperactivated as a result of a treatment such as immunotherapy. Examples can include lactate, iodoacetate, pyruvate, and various halopyruvates, including salts and acids thereof. Such molecules can function to inhibit cellular energy production in hyperactivated immune cells, thus limiting the ability of such cells to generate further cytokines.

In another example, the present disclosure provides systems and methods for treating a condition with combination of a therapy that generates detrimental amounts of cytokines and an immunoresponse regulator (i.e., energy inhibitor) to reduce the release of cytokines. Immunoresponse regulators function to inhibit energy production within immune cells that are releasing the detrimental level of cytokines. Examples can include lactate, iodoacetate, pyruvate, and various halopyruvates, including salts and acids thereof. Such molecules can function to inhibit cellular energy production in cytokine-releasing cells, thus limiting the ability of such cells to generate further cytokines. It is noted that, while the present disclosure refers to cytokines and cytokine hyperactivation, such does not limit the present scope.

Various conditions are contemplated that are treatable using immunotherapies that benefit from being combined with an immunoresponse regulator according to the present disclosure. Nonlimiting examples of such conditions include viral or other pathogenic conditions, immunodeficiencies, hypersensitivity reactions, autoimmune diseases, tissue and organ transplantations, cancers, inflammatory disorders, immunization reactions, among others. Specific nonlimiting examples include, myasthenia gravis, vasculitis, X-linked agammaglobulinemia, transient hypogammaglobulinemia of infancy, common variable immunodeficiency, severe combined immunodeficiency disease, selective immunoglobulin deficiencies, interstitial pneumonia in acquired immunodeficient states, hyper-IgM syndrome, lupus and lupus-like syndromes, recurrent viral infections in immunodeficiency syndromes, chronic mucocutaneous candidiasis, primary tuberculosis with immunodeficiency, Wiskott-Aldrich syndrome, chronic active hepatitis, coccidioidomycosis, Behcet disease, aphthous stomatitis, autoimmune polyendocrinopathy candidiasis ectodermal dystrophy, autoimmune lymphoproliferative syndrome, idiopathic CD4+ lymphocytopenia, complement system deficiencies, Chagas disease, lepromatous leprosy, HIV/AIDS, cryptococcal meningitis, septic shock, inflammatory bowel disease, ischemia-reperfusion injury, adult respiratory distress syndrome, osteoporosis, polyarteritis nodosa, glomerulonephritis, chronic granulomatous disease, marrow recovery following bone marrow transplantation, primary neutropenia, myelodysplasia, myeloproliferative disorders, aplastic anemia, and neutropenia associated with Felty syndrome, among others.

Additionally, various cancers are contemplated that are treatable using immunotherapies that benefit from being combined with an immunoresponse regulator according to the present disclosure. Nonlimiting examples include, bladder cancer, born cancer, breast cancer, cervical cancer, colon cancer, keratoacanthoma, kidney cancer, leukemia, liver cancer, lung cancer, lymphoma, medulloblastoma, melanoma, neuroblastoma, ovarian cancer, pancreatic cancer, prostate cancer, sarcoma, skin cancer, and the like, including combinations of such cancers.

When a white blood cell is hyperactivated, for example, ATP production is greatly increased. In such cases where white blood cells become hyperactivated and begin to damage uninfected tissue in a subject, sepsis and/or other detrimental processes can occur, often leading to significant damage/illness to the subject. By downregulating and/or killing these hyperactivated immune cells, the immunoresponse regulator can further reduce the damaging systemic effects that can occur.

One specific nonlimiting example of an immunoresponse regulator that is a useful cellular energy inhibitor is 3-bromopyruvate (3-BP). 3-BP is a small molecule that has sufficiently similar chemical structure to lactic acid that it enters hyperactivated cells through an upregulated lactic acid transport system. 3-BP has little effect on normal cells, as such cells contain very few lactic acid transporters when functioning in the normal state. Once in a hyperactivated immune cell, 3-BP damages glycolysis and oxidative phosphorylation systems due to its highly reactive nature, thus significantly reducing ATP production. This reduction in ATP production subsequently leads to the death of the hyperactivated immune cell and the eventual cessation of the effects produced by hyperactivation, such as the cytokine storm, for example.

Immunotherapy is a type of treatment that improves the immune system's ability to eliminate a given disease or condition. There are several types of immunotherapies, and each helps the immune system in a different way. The following describes immunotherapies used to treat cancer-however, it should be understood that the present scope extends to immunotherapies used to treat other conditions or diseases.

Cancer immunotherapy thus improves the immune system's ability to detect and/or eliminate cancer. The following provides a nonlimiting selection of cancer immunotherapies. As has been described, immunotherapy is defined as a treatment that uses a person's own immune system to detect and/or eliminate cancer. In other words, immunotherapy can boost or change how the immune system functions so it can find and attack cancer cells.

In one general type of immunotherapy used to treat cancer the immune system is stimulated to an increased activity in order to more readily and effectively find and eradicate cancer cells. In another general type of immunotherapy, components of the immune system are generated in vitro in a lab, which are then used to boost the immune system to a level whereby cancer cells can be more effectively found and eradicated.

### Adoptive Cellular Therapy

Adoptive cellular therapy is an example of a treatment that increases the number and/or effectiveness of immune cells, often T Cells, which improves the immune response against the cancer. There are at least four main types of adoptive cellular therapy:

Chimeric Antigen Receptor (CAR) T Cell therapy—The immune system recognizes foreign substances in the body by identifying antigens on the surface of such substances. T Cells have immune receptors that uniquely bind to foreign antigens, which activate other immune system components to begin the process of breaking down or otherwise eliminating the foreign substance associated with the foreign antigen. Because the antigens expressed by cancer cells may not be sufficiently foreign to bind immune receptors, T Cells often fail to initiate an immune response against them. In CAR T therapy, T Cells are collected from either a patient or donor and genetically engineered to express CARs that more closely match antigens being expressed by the targeted cancer. The CAR T cells are multiplied and infused back into the patient to increase the ability of T cells to recognize the cancer cells as foreign. Examples of cancers treated using CAR T therapies include, without limitation, leukemias, lymphomas, and multiple myelomas, including diffuse large B-cell lymphoma (DLBCL), follicular lymphoma, mantle cell lymphoma, B-cell acute lymphoblastic leukemia (ALL), and the like. Nonlimiting examples of specific CAR T-cell therapies include axicabtagene ciloleucel, brexucabtagene autoleucel, ciltacabtagene autoleucel, idecabtagene vicleucel, lisocabtagene maraleucel, and tisagenlecleucel.

As with many other cancer immunotherapies, CAR-T cell therapy can cause side effects, which can be severe. For example, increasing the number and activity of T cells in a subject increases the production of cytokines used as signals by the T cells. Cytokine-Release Syndrome (CRS) is a potentially serious condition that occurs when the cytokine signaling pathway is hyperactivated, leading to a continued increase in cytokine release that further stimulates an ever-increasing immune response. Cases of CRS can range from mild to life-threatening.

Chimeric Antigen Receptor (CAR) natural killer (NK) cell therapy—NK cells are immune system cells that identify and kill abnormal cells, including some cancer cells. Similar to CAR-T cell therapy, CAR-NK cell therapy is utilized to increase the detection ability of NK cells for a particular cancer, thus allowing the cancer cells to be detected as foreign.

Tumor infiltrating lymphocyte (TIL) therapy—TIL therapy involves collecting T cells from a portion of a patent's cancerous tumor, which recognize the cancer, but are too few in number to effectively. While such cells recognize the cancerous cells as foriegn, there are generally too few to combat the tumor. In TIL therapy, the number of T cells is increased in vitro and reintroduced back into the subject.

Endogenous T cell (ETC) therapy—in ETC therapy, T cells are extracted from a subject's blood, from which specific cancer-recognizing T cells are selected based on various biomarker profiles. These selected T cells are substantially increased in number and then reintroduced back into the subject.

### Cancer Vaccines

Cancer vaccines represent another form of cancer immunotherapy used to stimulate the immune system to recognize and fight specific types of cancer cells. Cancer vaccines often contain one of a) cancer cells taken from a subject's tumor, b) proteins designed to bind to cancer cells in order to increase recognition by the immune system, RNA vaccines that use the subject's cellular machinery to generate antigens associated with the cancer that stimulate the immune system against the cancerous cells, or d) proteins specific to a subject's tumor.

### Cytokine Therapy

In further detail of what has been described, cytokines are small proteins that cannot cross the lipid bilayer of cells and typically bind to cytokine receptors on the surfaces of target cells. They act through such cell surface receptors and play a crucial role in the immune system by modulating immune responses and regulating the maturation, growth, and responsiveness of specific cell populations. Additionally, cytokines can enhance or inhibit the action of other cytokines in response to infection, inflammation, trauma, sepsis, cancer, and the like. The cellular effects of cytokines depend on the particular cytokine, its extracellular abundance, the presence and abundance of the complementary receptor on the cell surface, and downstream signals that are activated by receptor binding.

A chemokine is one specific type of cytokine that influence immune cells to move toward a target. There are different kinds of chemokines, including interleukins, interferons, tumor necrosis factors, and growth factors.

Interleukins-Interleukins are a group of cytokines that act as chemical signals between white blood cells. Interleukin-2 (IL-2) helps immune system cells grow and divide more quickly. One version of IL-2 is used to treat advanced kidney cancer and metastatic melanoma. IL-2 can be used as a single drug treatment for these cancers, or it can be combined with chemotherapy or with other cytokines such as interferon-alfa. Other interleukins, such as IL-7, IL-12, and IL-21, continue to be studied for use against cancer too, both as adjuvants and as stand-alone agents.

Interferons-Interferons (IFNs) are proteins that facilitate the bodies resistance to infections and cancers. Various types of (IFN) includes IFN-alpha, IFN-beta, and IFN-gamma. Currently, IFN-alfa is used to treat cancer by boosting the ability of certain immune cells to attack cancer cells. It may also slow the growth of cancer cells directly, as well as the blood vessels that tumors need to grow. Cytokine therapy relies on interferons and interleukins to trigger an immune response within the subject. IL-2, for example, is used to treat kidney cancers and melanomas that have spread to other regions of the body. IFN-alpha is currently being used to treat melanoma, kidney cancer and certain leukemias and lymphomas.

Cytokine therapy, however, can cause adverse effects and has been linked to various disease states and conditions including schizophrenia, major depression, and Alzheimer's disease. T regulatory cells and associated cytokines Cytokine therapy has also been linked to can be engaged in the process of tumor immune escape, and may functionally inhibit immune response against the tumor.

An over-production of cytokines can additionally trigger the cytokine storm. The severity of the cytokine storm is weighted more heavily towards people with healthy immune systems due to their ability to produce stronger immune responses, which result in higher cytokine levels compared to those with compromised immune systems. It is believed that during the COVID-19 pandemic, the cytokine storm may have been the source of lung tissue damage and dysfunctional coagulation, among others.

### Monoclonal Antibodies

Antibodies are highly variable proteins that circulate through the blood stream and bind to the antigens foreign substances in order to signal immune cells to attack the foreign substances. A monoclonal antibody (mAb) is an antibody generated from cloned a white blood cell. Such mAbs generally have monovalent affinity, and thus bind to the same substate or epitope of the antigen recognized by the antibody. Monoclonal antibodies attach to specific proteins on the surface of cancer cells or immune cells and either 1) mark the cancer as a target for the immune system, or 2) boost the ability of immune cells to fight the cancer.

mAb treatments for cancer involve using mAbs that bind only to cancer-cell-specific antigens and induce an immune response against the target cancer cell. Such mAbs can be modified for delivery of a toxin, radioisotope, cytokine, or other active conjugate. Examples of mAbs approved by the FDA for the treatment of cancer include Alemtuzumab, Bevacizumab, Cetuximab, Dostarlimab, Gemtuzumab ozogamicin, Ipilimumab, Nivolumab, Ofatumumab, Panitumumab, Pembrolizumab, Ranibizumab, Rituximab, and Trastuzumab,

### Conjugated Monoclonal Antibodies

Conjugated mAb therapy, mAbs are combined with a chemotherapy drug or a radioactive particle. These mAbs are used as a homing device to take one of these substances directly to the cancer cells. The mAb circulates throughout the body until it can find and bind the target antigen, thereby delivering the toxic substance specifically to the targeted cells and lessening damage to normal cells in other parts of the body. Conjugated mAbs are also sometimes referred to as tagged, labeled, or loaded antibodies.

### Bispecific Monoclonal Antibodies

These drugs are made up of parts of 2 different mAbs, meaning they can bind to 2 different proteins at the same time. An example is blinatumomab (Blincyto), which is used to treat some types of leukemia. One part of blinatumomab attaches to the CD19 protein, which is found on some leukemia and lymphoma cells. Another part attaches to CD3, a protein found on immune cells called T cells. By binding to both of these proteins, this drug brings the cancer cells and immune cells together, which is thought to cause the immune system to attack the cancer cells.

Immune Checkpoint Inhibitors and their Side Effects

As has been described, one vital role of the immune system is the ability to detect foreign material (such as pathogens and cancer cells, for example) and to signal the presence of such. This allows the immune system to attack what is foreign while leaving normal cells alone. One mechanism facilitating this process of recognition is via “checkpoint” proteins on immune cells. The checkpoints function as molecular switches that need to be activated (or inactivated) to start an immune response. Some cancer cells, however, have the ability to bypass these checkpoints and avoid triggering an immune response.

Immune checkpoint therapy targets immune checkpoints, which are key regulators of the immune system. When stimulated, these checkpoint proteins can dampen the immune response to an immunologic stimulus, which is how some cancers protect themselves from attack. Checkpoint therapy can block inhibitory checkpoints, thus restoring immune system function. Immune checkpoint therapy thus helps cancer-fighting T cells to mount a longer-lasting response against the cancer.

Medicines that target different checkpoint proteins are now used to treat some types of cancer. Monoclonal antibodies, described above, are one class of drugs that can be used to target these checkpoint proteins. The following nonlimiting examples describe some of these drugs.

### PD-1 and PD-L1 Inhibitors

PD-1 is a checkpoint protein on T cells, which normally acts as an inhibitory control that helps keep T cells from indiscriminately attacking other cells in the body. It does this by binding to PD-L1, a protein expressed on normal (and some cancer) cells. When PD-1 binds to PD-L1, it signals “normal” to T cells, which as a result, do not attack the cell. Some cancer cells overexpress PD-L1, thus appearing to T cells as normal cells. Monoclonal antibodies that target either PD-1 or PD-L1 can block this binding and boost the immune response against cancer cells.

Both PD-1 and PD-L1 inhibitors have been shown to be helpful in treating many different types of cancer. Examples of drugs that target PD-1 include Pembrolizumab (Keytruda), Nivolumab (Opdivo), and Cemiplimab (Libtayo). Examples of drugs that target PD-L1 include Atezolizumab (Tecentriq), Avelumab (Bavencio), and Durvalumab (Imfinzi).

### CTLA-4 Inhibitors

CTLA-4 is another checkpoint protein on some T cells that acts as a type of “off switch” to help keep the immune system in check. Ipilimumab (Yervoy) is a monoclonal antibody that binds CTLA-4 and removes this inhibitory check, thus boosting the immune response to the cancer. This drug is typically used along with a PD-1 inhibitor, such as nivolumab. It can be used to treat melanoma of the skin and several other types of cancer.

### LAG-3 Inhibitors

LAG-3 is another checkpoint protein on some types of immune cells that normally acts as a type of “off switch” to help keep the immune system in check. Relatlimab is a monoclonal antibody that binds LAG-3 and removes this inhibitory check, thus boosting the immune response to the cancer. This drug is given along with the PD-1 inhibitor nivolumab (in a combination known as Opdualag). It can be used to treat melanoma of the skin, and it's being studied for use in several other types of cancer.

By targeting a checkpoint protein, these and similar drugs remove one of the safeguards on the body's immune system. Sometimes the immune system responds to by attacking other parts of the body, which can cause serious or even life-threatening problems in the lungs, intestines, liver, hormone-making glands, kidneys, or other organs. This process of the immune system attacking the organs and tissues of the body is known as an autoimmune reaction. By using an immunoresponse regulator to remove such checkpoints (e.g., 3-bromopyruvate as disclosed herein), hyperactivated immune cells are eliminated before an autoimmune reaction can occur. It is additionally noted that autoimmune reactions can occur with other immunotherapies, and as such, prevention or reduction of autoimmune reactions in association with other immunotherapies is considered to be within the present scope.

### Immunomodulators

Immunomodulators are a group of drugs that mainly target the pathways that treat multiple myeloma and a few other cancers. They function in various ways, including working on the immune system directly by turning down some proteins and turning up others.

### Immunoresponse Regulator/Cellular Energy Inhibitor

Generally, there are two energy (ATP) production factories inside cells, i.e., glycolysis and oxidative phosphorylation by mitochondria. In normal cells, about 5% of the total cellular energy (ATP) production is derived from glycolysis and about 95% from the mitochondria. In hyperactivated immune cells, the energy production by glycolysis can be significantly increased (up to 60%). This dramatic increase in glycolysis results in a significant increase in lactic acid production.

Examples of cellular energy inhibitors include lactate, iodoacetate, pyruvate, and a halopyruvate, including salts and acids thereof. In one specific example, the presently disclosed cellular energy inhibitor (immunoresponse regulator) is 3-bromopyruvate (3-BP) (a lactic acid analog).

As described above, 3-BP is a small molecule that has sufficiently similar chemical structure to lactic acid that it can enter hyperactivated cells through an upregulated lactic acid transport system. 3BP has little effect on normal cells, as such cells contain very few lactic acid transporters when functioning in the normal state. Once in a hyperactivated immune cell, 3-BP damages glycolysis and oxidative phosphorylation systems due to its highly reactive nature, thus significantly reducing ATP production. This reduction in ATP production subsequently leads to the death of the hyperactivated immune cell and the eventual cessation of the effects produced by hyperactivation, such as the cytokine storm, for example.

In addition to functioning as immunoresponse regulators, 3-halopyruvates such as 3-BP, for example, can function as immunotherapy agents. This dual function allows 3-BP to act as an immunoresponse regulator of the effects of a hyperactivated immune system and an additional immunotherapy.

In one example, 3-BP has an immunotherapeutic effect on the PD-1/PD-L1 immune checkpoint (described above) by disrupting the binding of PD-1 to PD-L1. Without intending to be bound by any scientific theory, 3-BP binds to PD-1 with a sufficiently high affinity to preclude PD-1 binding with PD-L1. As described above, PD-1/PD-L1 binding generates an inhibitory effect by signaling T-cells to not attack the cell, as it is “self” as opposed to “foreign invader.” The binding of 3-BP to PD-1 thus removes the inhibitory control, causing T-cells to recognize the cancer cells as foreign and attack them as such.

Furthermore, 3-BP has a direct therapeutic effect on cancer cells due to its ability to inhibit energy production inside these cells. Once energy production is inhibited, the cells begin to die and release cellular contents into the tissue, which causes a localized immune response, which in turn heightens the immune recognition of the remaining cancer cells.

In one specific example, the cellular energy inhibitor can be a molecule according to Formula I:

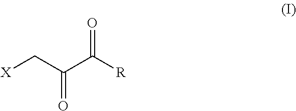

Various specific molecules are contemplated, wherein, for example, X can be, without limitation, a nitro, an imidazole, a halide, sulfonate, a carboxylate, an alkoxide, amine oxide, or the like. Additionally, R can be, without limitation, OR′, N(R″)₂, C(O)R′″, C1-C6 alkyl, C6-C12 aryl, C1-C6 heteroalkyl, a C6-C12 heteroaryl, H, an alkali metal or the like, where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl.

In some examples, the cellular energy inhibitor composition can include a variety of excipients, active agents, prodrugs, metabolites, buffers, and the like, such as, for example, one or more sugars, polyalcohols, or the like, glycolysis inhibitors, biological buffers, and the like. In some examples the cellular energy inhibitor molecule can be formulated in a composition with at least one sugar, which can stabilize the cellular energy inhibitor by substantially preventing the inhibitor from hydrolyzing.

In one example, R of formula (I) can be OH and X of formula (I) can be a nitro, an imidazole, a halide, a sulfonate, a carboxylate, an alkoxide, an amine oxide, or the like. Additionally, X can be a halide, such as, for example, fluoride, bromide, chloride, iodide, or the like. In one example, X can be a sulfonate, such as, for example, a triflate, a mesylate, a tosylate, or the like. In another example, X can be amine oxide. In still another example, the amine oxide can be dimethylamine oxide.

In another example, the cellular energy inhibitor can be a 3-halopyruvate, such as, for example, 3-fluoropyruvate, 3-chloropyruvate, 3-bromopyruvate, 3-iodopyruvate, or a combination thereof. A general structure showing a halide in the 3-position is shown in formula II.

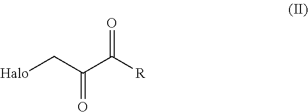

In a further nonlimiting example, the cellular energy inhibitor can have bromine in the 3-position, as shown in formula III.

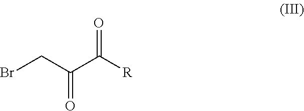

In one further nonlimiting example, the cellular energy inhibitor can be 3-bromopyruvate (3-BP), as shown in formula IV.

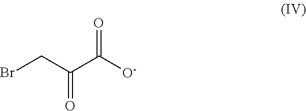

In another nonlimiting example, the cellular energy inhibitor can be 3-bromopyruvic acid, as shown in formula V.

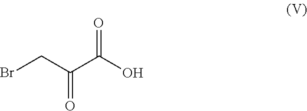

It is noted that 3-bromopyruvate or 3-bromopyruvic acid can be referred to herein as “3-BP,” and that the two molecules can be used interchangeably unless the context clearly indicates otherwise.

In some examples, the cellular energy inhibitor can be formulated in a composition with at least one sugar, which can stabilize the cellular energy inhibitor by substantially preventing the cellular energy inhibitor from hydrolyzing. In some examples, a composition can include 3-BP, as a cellular energy inhibitor, for example, and at least one sugar, at least two sugars, at least three sugars, and the like. In one example, a sugar can include a monosaccharide, a disaccharide, an oligosaccharide, or a combination thereof. Nonlimiting examples of monosaccharides can include glucose, fructose, galactose, and the like. Nonlimiting examples of disaccharides can include sucrose, lactose, maltose, and the like. It is noted that, for the purposes of the present disclosure, the term “sugar” can also include oligosaccharides, polysaccharides, polyols, polyalcohols, and similar molecules that function to stabilize 3-BP.

A sugar can include a 3-carbon sugar, a 4-carbon sugar, a 5-carbon sugar, a 6-carbon sugar, a 7-carbon sugar, and the like, including combinations thereof. In one aspect, the sugar can be a 3-carbon sugar, a 4-carbon sugar, a 5-carbon sugar, a 6-carbon sugar, a 7-carbon sugar, and the like, including combinations thereof, provided the sugar is not involved in energy metabolism to the extent that it generates energy (i.e., a nonmetabolizable sugar).

In one example, the sugar can be gluconic acid. In another example, the sugar can be glucuronic acid. At least one of the sugars can be a five-carbon sugar. In one example, at least two of the sugars can be five-carbon sugars. The five-carbon sugars can be independently selected from mannitol, erythritol, isomalt, lactitol, maltitol, sorbitol, xylitol, dulcitol, ribitol, inositol, or the like, including combinations thereof. In one example, at least one of the sugars can be glycerol. In another example, the sugars can be glycerol, inositol, and sorbitol. Other nonlimiting example of sugars can include ethylene glycol, threitol, arabitol, galactitol, fucitol, iditol, volemitol, maltotriitol, maltotetraitol, and polyglycitol, including combinations thereof. In one example, the sugars can include glycerol, inositol, sorbitol, mannitol or any combination thereof. In another example, the sugars can include glycerol, inositol, sorbitol, or any combination thereof. In yet another example, the inositol can be myo-inositol. In other examples, the sugar can be a polyalcohol.

The sugars described herein can be any isomeric form. In one example, the compositions described herein can include the less biologically active form of the sugar as compared to its isomer. In one case, the less biologically active sugar can be the L-enantiomer sugar. However, if the D-enantiomer sugar is found to be less biologically active as compared to its L form, then the D form can be used. In one example, such sugars can function as a glycolytic inhibitor.

In one example, a composition can include one or more sugars in a range from about 0.5 wt % to about 50.0 wt % or from about 1.0 wt % to about 25.5 wt %. In yet another example, a composition can include one or more sugars in a range from about 0.2 wt % to about 75.0 wt % or from about 0.5 wt % to about 50.0 wt %. In a further example, a composition can include one or more sugars in a range from about 0.1 wt % to about 25.0 wt %, from about 0.2 wt % to about 10.0 wt %.

In some examples, the composition can include glycerol in a range from about 0.1 wt % to about 5.0 wt % or from about 0.1 wt % to about 3.0 wt %. In other examples, the composition can include inositol in a range from about 0.1 wt % to about 10 wt %, from about 0.1 wt % to about 6 wt %. In further examples, the composition can include sorbitol in a range from about 0.1 wt % to about 40.0 wt % or from about 0.1 wt % to about 30 wt %. In yet further examples, the composition can include mannitol in a range from about 0.1 wt % to about 30 wt % or from about 0.1 wt % to about 10 wt %. Additionally, each of the sugars may be added in a volume up to a maximum solubility of the sugar in the formulation or composition. It is additionally noted that the above wt % s of ingredients are without water or other liquid carrier. Additionally, each of the sugars may be added in a volume up to a maximum solubility of the sugar in the formulation or composition.

Generally, 3-BP can be formulated as any type of dosage form capable of being delivered to a subject. Such dosage forms can be enteral, parenteral, transdermal, or the like. Enteral dosage forms can be sustained release or immediate release and can include, without limitation, tablets, lozenges, capsules, caplets, encapsulated pellets, encapsulated granules, encapsulated powders, gelatin capsules, liquids, syrups, elixirs, suspensions, sprays, aerosols, powders, and the like, including combinations thereof. Nonlimiting examples of transdermal dosage forms can include lotions, gels, creams, pastes, ointments, liquid sprays, liquid drops, powder sprays, wipes, emulsions, aerosols, transmucosal tablets, adhesive devices, adhesive matrix-type transdermal patches, liquid reservoir transdermal patches, microneedle devices, magnetic devices, and the like. Nonlimiting examples of parenteral dosage forms can include intravenous, subcutaneous, and the like.

In some examples, a 3-BP composition can include a biological buffer that is present in an amount sufficient to at least partially deacidify the cellular energy inhibitor and neutralize metabolic by-products of the cellular energy inhibitor. Nonlimiting examples of biological buffers can include a citrate buffer, a phosphate buffer, an acetate buffer, and the like, including combinations thereof. In one specific example, the biological buffer can be a citrate buffer, such as, without limitation, sodium citrate. In another specific example, the biological buffer can be a phosphate buffer, such as, without limitation, sodium phosphate. In one specific example, the biological buffer can be an acetate buffer, such as, without limitation, sodium acetate. In yet other examples, the biological buffer can include at least two biological buffers, such as, without limitation, a citrate buffer and an acetate buffer, a citrate buffer and a phosphate buffer, an acetate buffer and a phosphate buffer, or a citrate buffer, a phosphate buffer, and an acetate buffer.

In some examples, the composition can comprise the biological buffer in a concentration of from about 0.1 mM to about 200 mM. In one embodiment, the composition can comprise the biological buffer in a concentration of from about 1 mM to about 20 mM. In some examples, the composition can include the biological buffer in a range of from about 0.1 wt % to about 15 wt % or from about 2.0 wt % to about 8.0 wt %. Additionally, the biological buffer can maintain a physiological pH of 4.0 to 8.5. In one embodiment, the biological buffer can maintain a physiological pH of 5.5 to 8.0. In another embodiment, the biological buffer can maintain a physiological pH of 6.8 to 7.8. In still another embodiment, the biological buffer can maintain a physiological pH of 7.3 to 7.6. It is additionally noted that the above wt % s of ingredients are without water or other liquid carrier.

In some examples, the present 3-BP formulations can comprise antifungal agents, antibiotics, glycolysis inhibitors, inhibitors of mitochondria, sugars, and biological buffers, without limitation. Examples of such agents include, but are not limited to, amphotericin B, efrapeptin, doxorubicin, (2-DG), analogs of 2-DG, d-lactic acid, dichloroacetic acid (or salt form of dichloroacetate), oligomycin, analogs of oligomycin, glycerol, inositol, sorbitol, glycol, erythritol, threitol, arabitol, xylitol, ribitol, mannitol, dulcitol, iditol, isomalt, maltitol, lactitol, polyglycitol, sodium phosphate, sodium citrate, sodium acetate, sodium carbonate, sodium bicarbonate, sodium pyruvate, sodium lactate, oxaloacetate, isocitrate, aconitate, succinate, fumarate, malate, diluted saline solutions with varying concentrations of NaCl, and water. In addition to the sodium ion that accompanies these biological buffers, calcium and potassium cations can also accompany the biological buffers. Various active agents of the composition can include a cellular energy inhibitor, a glycolysis inhibitor, a mitochondria inhibitor, a halo monocarboxylate compound, an antifungal agent, an antibiotic agent, and the like. In the various dosage forms described above, any of the above ingredients can be included with 3-BP, any of the excipients, or in a separate vessel.

In addition to the above components, the 3-BP compositions described herein can further comprise a halo monocarboxylate compound that is separate from the cellular energy inhibitor. In cases where the halo monocarboxylate compound can function to inhibit glycolysis and/or mitochondria function, the halo monocarboxylate can be considered a second cellular energy inhibitor. In one embodiment, the halo monocarboxylate compound can be a halo two-carbon monocarboxylate compound. The halo two-carbon monocarboxylate compound can be selected from, without limitation, 2-fluoroacetate, 2-chloroacetate, 2-bromoacetate, 2-iodoacetate, and the like, including combinations thereof. In one embodiment, the halo two-carbon monocarboxylate compound can be 2-bromoacetate. In one example, the composition can comprise the halo two-carbon monocarboxylate compound in a concentration from about 0.01 mM to about 5.0 mM. In another example, the composition can comprise a halo two-carbon monocarboxylate compound in a concentration from about 0.1 mM to about 0.5 mM.

Additionally, a halo monocarboxylate compound can be a halo three-carbon monocarboxylate compound. In one embodiment, the halo three-carbon monocarboxylate compound can be selected from, without limitation, 3-fluorolactate, 3-chlorolactate, 3-bromolactate, 3-iodolactate, and the like, including combinations thereof. In another example, the composition can include the halo three-carbon monocarboxylate compound in a concentration from about 0.5 mM to about 250 mM. In one embodiment, the composition can comprise the halo three-carbon monocarboxylate compound in a concentration from about 10 mM to about 50 mM.

In some examples, the 3-BP formulations described herein can further comprise a mitochondrial inhibitor in addition to the cellular energy inhibitor. The mitochondrial inhibitor can be selected from, without limitation, oligomycin, efrapeptin, aurovertin, and the like, including combinations thereof. In another example, the composition can include the mitochondrial inhibitor in a concentration from about 0.001 mM to about 5.0 mM. In one example, the composition can include the mitochondrial inhibitor in a concentration from about 0.01 mM to about 0.5 mM.

In some examples, the present 3-BP compositions described herein can further comprise an antifungal agent and/or antibacterial agent. In one embodiment, the composition can individually comprise the antifungal agent and/or antibacterial agent in a concentration from about 0.01 mM to about 5.0 mM. In another embodiment, the composition can individually comprise the antifungal agent and/or antibacterial agent in a concentration from about 0.05 mM to about 0.5 mM.

In some examples, a 3-BP formulation can include a glycolysis inhibitor. Many suitable glycolysis inhibitors are contemplated, however a nonlimiting list can include 2-DG, lonidamine, imatinib, oxythiamine, 6-aminonicotinamide, genistein, 5-thioglucose (5-TG), mannoheptulose, α-chlorohydrin, oridazole, oxalate, glufosfamide, and the like, including combinations thereof. The 3-BP formulation can include the glycolysis inhibitor in any effective amount.

In addition to the above concentrations, the present compositions can have various ratios of the components described herein. In one embodiment, the cellular energy inhibitor and biological buffer can be present in a ratio ranging from 1:1 to 1:5 by mM. In another embodiment, the cellular energy inhibitor and glycolysis inhibitor can be present in a ratio ranging from 5:1 to 1:1 by mM. In still another embodiment, the cellular energy inhibitor and the at least one sugar are present in a ratio ranging from 1:1 to 1:5 by mM. In yet another embodiment, the cellular energy inhibitor and the halo two-carbon monocarboxylate compound can be present in a ratio ranging from 20:1 to 4:1 by mM. In still yet another embodiment, the cellular energy inhibitor to mitochondrial inhibitor can be present in a ratio ranging from 20:1 to 40:1 by mM.

In some examples, the 3-BP compositions described herein can further include a hexokinase inhibitor. The hexokinase inhibitor can be any molecule that inhibits hexokinase 1, hexokinase 2, and/or any isozyme thereof (collectively referred to herein as “hexokinase”). As used herein, “hexokinase 1” or “hexokinase 1 isozyme” refers to any isoforms of hexokinase 1 and its naturally known variants, including those provided in SEQ ID NO: 1, SEQ ID NO: 2, SEQ ID NO: 3, and SEQ ID NO: 4, as follows:

| (SEQ ID NO: 1) | |
|---|---|
| 1 | MIAAQLLAYY FTELKDDQVK KIDKYLYAMR LSDETLIDIM TRFRKEMKNG LSRDFNPTAT |
| 61 | VKMLPTFVRS IPDGSEKGDF IALDIGGSSF RILRVQVNHE KNQNVHMESE VYDTPENIVH |
| 121 | GSGSQLFDHV AECLGDFMEK RKIKDKKLPV GFTFSFPCQQ SKIDEAILIT WTKRFKASGV |
| 181 | EGADVVKLLN KAIKKRGDYD ANIVAVVNDT VGTMMTCGYD DQHCEVGLII GTGTNACYME |
| 241 | ELRHIDLVEG DEGRMCINTE WGAFGDDGSL EDIRTEFDRE IDRGSLNPGK QLFEKMVSGM |
| 301 | YLGELVRLIL VKMAKEGLLF EGRITPELLT RGKFNTSDVS AIEKNKEGLH NAKEILTRLG |
| 361 | VEPSDDDCVS VQHVCTIVSF RSANLVAATL GAILNRLRDN KGTPRLRTTV GVDGSLYKTH |
| 421 | PQYSRRFHKT LRRLVPDSDV RFLLSESGSG KGAAMVTAVA YRLAEQHRQI EETLAHFHLT |
| 481 | KDMLLEVKKR MRAEMELGLR KQTHNNAVVK MLPSFVRRTP DGTENGDFLA LDLGGTNFRV |
| 541 | LLVKIRSGKK RTVEMHNKIY AIPIEIMQGT GEELFDHIVS CISDFLDYMG IKGPRMPLGF |
| 601 | TFSFPCQQTS LDAGILITWT KGFKATDCVG HDVVTLLRDA IKRREEFDLD VVAVVNDTVG |
| 661 | TMMTCAYEEP TCEVGLIVGT GSNACYMEEM KNVEMVEGDQ GQMCINMEWG AFGDNGCLDD |
| 721 | IRTHYDRLVD EYSLNAGKQR YEKMISGMYL GEIVRNILID FTKKGFLFRG QISETLKTRG |
| 781 | IFETKFLSQI ESDRLALLQV RAILQQLGLN STCDDSILVK TVCGVVSRRA AQLCGAGMAA |
| 841 | VVDKIRENRG LDRLNVTVGV DGTLYKLHPH FSRIMHQTVK ELSPKCNVSF LLSEDGSGKG |
| 901 | AALITAVGVR LRTEASS |
| (SEQ ID NO: 2) | |
| 1 | MDCEHSLSLP CRGAEAWEIG IDKYLYAMRL SDETLIDIMT RFRKEMKNGL SRDFNPTATV |
| 61 | KMLPTFVRSI PDGSEKGDFI ALDLGGSSFR ILRVQVNHEK NQNVHMESEV YDTPENIVHG |
| 121 | SGSQLFDHVA ECLGDFMEKR KIKDKKLPVG FTFSFPCQQS KIDEAILITW TKRFKASGVE |
| 181 | GADVVKLLNK AIKKRGDYDA NIVAVVNDTV GTMMTCGYDD QHCEVGLIIG TGTNACYMEE |
| 241 | LRHIDLVEGD EGRMCINTEW GAFGDDGSLE DIRTEFDREI DRGSLNPGKQ LFEKMVSGMY |
| 301 | LGELVRLILV KMAKEGLLFE GRITPELLTR GKFNTSDVSA IEKNKEGLHN AKEILTRLGV |
| 361 | EPSDDDCVSV QHVCTIVSFR SANLVAATLG AILNRLRDNK GTPRLRTTVG VDGSLYKTHP |
| 421 | QYSRRFHKTL RRLVPDSDVR FLLSESGSGK GAAMVTAVAY RLAEQHRQIE ETLAHFHLTK |
| 481 | DMLLEVKKRM RAEMELGLRK QTHNNAVVKM LPSFVRRTPD GTENGDFLAL DLGGTNFRVL |
| 541 | LVKIRSGKKR TVEMHNKIYA IPIEIMQGTG EELFDHIVSC ISDFLDYMGI KGPRMPLGFT |
| 601 | FSFPCQQTSL DAGILITWTK GFKATDCVGH DVVTLLRDAI KRREEFDLDV VAVVNDTVGT |
| 661 | MMTCAYEEPT CEVGLIVGTG SNACYMEEMK NVEMVEGDQG QMCINMEWGA FGDNGCLDDI |
| 721 | RTHYDRLVDE YSLNAGKQRY EKMISGMYLG EIVRNILIDF TKKGFLFRGQ ISETLKTRGI |
| 781 | FETKFLSQIE SDRLALLQVR AILQQLGLNS TCDDSILVKT VCGVVSRRAA QLCGAGMAAV |
| 841 | VDKIRENRGL DRLNVTVGVD GTLYKLHPHF SRIMHQTVKE LSPKCNVSFL LSEDGSGKGA |
| 901 | ALITAVGVRL RTEASS |
| (SEQ ID NO: 3) | |
| 1 | MGQICQRESA TAAEKPKLHL LAESEIDKYL YAMRLSDETL IDIMTRFRKE MKNGLSRDFN |
| 61 | PTATVKMLPT FVRSIPDGSE KGDFIALDLG GSSFRILRVQ VNHEKNQNVH MESEVYDTPE |
| 121 | NIVHGSGSQL FDHVAECLGD FMEKRKIKDK KLPVGFTFSF PCQQSKIDEA ILITWTKRFK |
| 181 | ASGVEGADVV KLLNKAIKKR GDYDANIVAV VNDTVGTMMT CGYDDQHCEV GLIIGTGTNA |
| 241 | CYMEELRHID LVEGDEGRMC INTEWGAFGD DGSLEDIRTE FDREIDRGSL NPGKQLFEKM |
| 301 | VSGMYLGELV RLILVKMAKE GLLFEGRITP ELLTRGKFNT SDVSAIEKNK EGLHNAKEIL |
| 361 | TRLGVEPSDD DCVSVQHVCT IVSFRSANLV AATLGAILNR LRDNKGTPRL RTTVGVDGSL |
| 421 | YKTHPQYSRR FHKTLRRLVP DSDVRFLLSE SGSGKGAAMV TAVAYRLAEQ HRQIEETLAH |
| 481 | FHLTKDMLLE VKKRMRAEME LGLRKQTHNN AVVKMLPSFV RRTPDGTENG DFLALDLGGT |
| 541 | NFRVLLVKIR SGKKRTVEMH NKIYAIPIEI MQGTGEELFD HIVSCISDFL DYMGIKGPRM |
| 601 | PLGFTFSFPC QQTSLDAGIL ITWTKGFKAT DCVGHDVVTL LRDAIKRREE FDLDVVAVVN |
| 661 | DTVGTMMTCA YEEPTCEVGL IVGTGSNACY MEEMKNVEMV EGDQGQMCIN MEWGAFGDNG |
| 721 | CLDDIRTHYD RLVDEYSLNA GKQRYEKMIS GMYLGEIVRN ILIDFTKKGF LFRGQISETL |
| 781 | KTRGIFETKF LSQIESDRLA LLQVRAILQQ LGLNSTCDDS ILVKTVCGVV SRRAAQLCGA |
| 841 | GMAAVVDKIR ENRGLDRLNV TVGVDGTLYK LHPHFSRIMH QTVKELSPKC NVSFLLSEDG |
| 901 | SGKGAALITA VGVRLRTEAS S |
| (SEQ ID NO: 4) | |
| 1 | MAKRALRDFI DKYLYAMRLS DETLIDIMTR FRKEMKNGLS RDFNPTATVK MLPTFVRSIP |
| 61 | DGSEKGDFIA LDLGGSSFRI LRVQVNHEKN QNVHMESEVY DTPENIVHGS GSQLFDHVAE |
| 121 | CLGDFMEKRK IKDKKLPVGF TFSFPCQQSK IDEAILITWT KRFKASGVEG ADVVKLINKA |
| 181 | IKKRGDYDAN IVAVVNDTVG TMMTCGYDDQ HCEVGLIIGT GTNACYMEEL RHIDLVEGDE |
| 241 | GRMCINTEWG AFGDDGSLED IRTEFDREID RGSLNPGKQL FEKMVSGMYL GELVRLILVK |
| 301 | MAKEGLLFEG RITPELLTRG KFNTSDVSAI EKNKEGLHNA KEILTRLGVE PSDDDCVSVQ |
| 361 | HVCTIVSFRS ANLVAATLGA ILNRLRDNKG TPRLRTTVGV DGSLYKTHPQ YSRRFHKTLR |
| 421 | RLVPDSDVRF LLSESGSGKG AAMVTAVAYR LAEQHRQIEE TLAHFHLTKD MLLEVKKRMR |
| 481 | AEMELGLRKQ THNNAVVKML PSFVRRTPDG TENGDFLALD LGGTNFRVLL VKIRSGKKRT |
| 541 | VEMHNKIYAI PIEIMQGTGE ELFDHIVSCI SDFLDYMGIK GPRMPLGFTF SFPCQQTSLD |
| 601 | AGILITWTKG FKATDCVGHD VVTLLRDAIK RREEFDLDVV AVVNDTVGTM MTCAYEEPTC |
| 661 | EVGLIVGTGS NACYMEEMKN VEMVEGDQGQ MCINMEWGAF GDNGCLDDIR THYDRLVDEY |
| 721 | SLNAGKQRYE KMISGMYLGE IVRNILIDFT KKGFLFRGQI SETLKTRGIF ETKFLSQIES |
| 781 | DRLALLQVRA ILQQLGLNST CDDSILVKTV CGVVSRRAAQ LCGAGMAAVV DKIRENRGLD |
| 841 | RLNVTVGVDG TLYKLHPHFS RIMHQTVKEL SPKCNVSFLL SEDGSGKGAA LITAVGVRLR |
| 901 | TEASS |

As used herein, “hexokinase 2” or “hexokinase 2 isozyme” refers to any isoforms of hexokinase 2 and its naturally known variants, including that provided in SEQ ID NO: 5 as follows:

| (SEQ ID NO: 5) | |
|---|---|
| 1 | MIASHLLAYF FTELNHDQVQ KVDQYLYHMR LSDETLLEIS KRFRKEMEKG LGATTHPTAA |
| 61 | VKMLPTFVRS TPDGTEHGEF LALDLGGTNF RVLWVKVTDN GLQKVEMENQ IYAIPEDIMR |
| 121 | GSGTQLFDHI AECLANFMDK LQIKDKKLPL GFTFSFPCHQ TKLDESFLVS WTKGFKSSGV |
| 181 | EGRDVVALIR KAIQRRGDFD IDIVAVVNDT VGTMMTCGYD DHNCEIGLIV GTGSNACYME |
| 241 | EMRHIDMVEG DEGRMCINME WGAFGDDGSL NDIRTEFDQE IDMGSLNPGK QLFEKMISGM |
| 301 | YMGELVRLIL VKMAKEELLF GGKLSPELLN TGRFETKDIS DIEGEKDGIR KAREVLMRLG |
| 361 | LDPTQEDCVA THRICQIVST RSASLCAATL AAVLQRIKEN KGEERLRSTI GVDGSVYKKH |
| 421 | PHFAKRLHKT VRRLVPGCDV RFLRSEDGSG KGAAMVTAVA YRLADQHRAR QKTLEHLQLS |
| 481 | HDQLLEVKRR MKVEMERGLS KETHASAPVK MLPTYVCATP DGTEKGDFLA LDLGGTNFRV |
| 541 | LLVRVRNGKW GGVEMHNKIY AIPQEVMHGT GDELFDHIVQ CIADFLEYMG MKGVSLPLGF |
| 601 | TFSFPCQQNS LDESILLKWT KGFKASGCEG EDVVTLLKEA IHRREEFDLD VVAVVNDTVG |
| 661 | TMMTCGFEDP HCEVGLIVGT GSNACYMEEM RNVELVEGEE GRMCVNMEWG AFGDNGCLDD |
| 721 | FRTEFDVAVD ELSLNPGKQR FEKMISGMYL GEIVRNILID FTKRGLLFRG RISERLKTRG |
| 781 | IFETKFLSQI ESDCLALLQV RAILQHLGLE STCDDSIIVK EVCTVVARRA AQLCGAGMAA |
| 841 | VVDRIRENRG LDALKVTVGV DGTLYKLHPH FAKVMHETVK DLAPKCDVSF LQSEDGSGKG |
| 901 | AALITAVACR IREAGQR |

As has been described, a major source of ATP production occurs in mitochondria in normal cells. However, ATP production from glycolysis is significantly upregulated in cancer cells. One reason for this upregulation is due to hexokinase molecules binding to, and forming complexes with, mitochondrial voltage dependent anion channels (VDACs) at ATP synthasomes, thus forming so called “ATP synthasome mega complexes.” The formation of such ATP synthasome mega complexes can immortalize the cancer cell, thus allowing the continued use of the cell's energy production processes for cancer growth. A hexokinase inhibitor, therefore, can thus block hexokinase from binding to the VADCs or displace hexokinase molecules from the VADCs of already formed ATP synthasome mega complexes.

In one example, a hexokinase inhibitor can be up to 25 amino acid units from the N-terminal region of Hexokinase 2 isozyme or Hexokinase 1 isozyme. In another example, the hexokinase inhibitor can be an amino acid sequence of 5 to 20 amino acid units, where the 5 to 20 amino acid sequence is present in the first 25 amino acid unit region beginning from the N-terminal end of hexokinase 1 isozyme or hexokinase 2 isozyme. In one example, the 5 to 20 amino acid sequence can be any 5-20 amino acid sequence present in the first 25 amino acid unit region of the N-terminus of Hexokinase 1 1 or Hexokinase 2. Such amino acid sequences can displace cellular bound hexokinase or competitively bind to voltage dependent anion channels (VDAC), thus preventing initial hexokinase binding.

In other examples, a hexokinase inhibitor can include antibodies against a portion of HK1 or HK2, such as, for example, the N-terminal region of either molecule. In one specific example, a hexokinase inhibitor can be an amino acid sequence, such as SEQ ID NO: 6, corresponding to the first 25 amino acids from the N-terminus end of hexokinase 1 (isoform1) having a sequence as follows:

| (SEQ ID NO: 6) | |
|---|---|
| 1 | MIAAQLLAYY FTELKDDQVK KIDKY |

In another example, a hexokinase inhibitor can be an amino acid sequence as in SEQ ID NO: 7, corresponding to the first 25 amino acids from the N-terminus end of hexokinase 1 (isoform 2) having a sequence as follows:

| (SEQ ID NO: 7) | |
|---|---|
| 1 | MDCEHSLSLP CRGAEAWEIG IDKYL |

In yet another example, a hexokinase inhibitor can be an amino acid sequence as in SEQ ID NO: 8, corresponding to the first 25 amino acids from the N-terminus end of hexokinase 1 (isoform 3) having a sequence as follows:

| (SEQ ID NO: 8) | |
|---|---|
| 1 | MGQICQRESA TAAEKPKLHL LAESE |

In still another example, a hexokinase inhibitor can be an amino acid sequence as in SEQ ID NO: 9, corresponding to the first 25 amino acids from the N-terminus end of hexokinase 1 (isoform 4) having a sequence as follows:

| (SEQ ID NO: 9) | |
|---|---|
| 1 | MAKRALRDFI DKYLYAMRLS DETLI |

In yet another example, a hexokinase inhibitor can be an amino acid sequence as in SEQ ID NO: 10, corresponding to the first 25 amino acids from the N-terminus end of hexokinase 2 having a sequence as follows:

(SEQ ID NO: 10)
MIASHLLAYF FTELNHDQVQ KVDQY

Additional hexokinase inhibitors can be those as disclosed in U.S. Pat. No. 5,854,067 (to Newgard et al, issued Dec. 29, 1998) and/or U.S. Pat. No. 5,891,717 (to Newgard et al., issued Apr. 6, 1999), both of which are incorporated by reference in their entireties. Additional hexokinase inhibitors that can be used in the present formulations include those disclosed in U.S. Pat. Nos. 6,670,330; 6,218,435; 5,824,665; 5,652,273; and 5,643,883; and U.S. patent application publication Nos. 20030072814; 20020077300; and 20020035071; each of the foregoing patent publications and patent application is incorporated herein by reference, in their entireties.

In some examples, the 3-BP compositions described herein can further comprise various ingredients recited below. In the various dosage forms described above in FIGS. 4-7, any of these various ingredients can be admixed with the 3-BP, provided they are nonreactive therewith, or be present in a separate layer or in any layer described above, provided the ingredient(s) is/are reactively isolated in the storage form.

In one embodiment, the present compositions can include less biologically active amino acids as compared to their isomers to facilitate cancer cell starvation. In one aspect, the less biologically active amino acid can be a D-amino acid. However, if the L-amino acid is less biologically active than the D-form, the L-amino acid can be used.

In one embodiment, the present compositions can include inhibitors for DNA replication; inhibitors for DNA binding; and/or inhibitors for DNA transcription. In another embodiment, the present compositions can include inhibitors for cell cycle, growth and/or proliferation. In yet another embodiment, the present compositions can include inhibitors for signal transduction pathways. In yet another embodiment, the present compositions can include inhibitors for angiogenesis. In yet another embodiment, the present compositions can include small RNAs that interfere with normal gene control including antisense RNA, micro RNA, small hairpin RNA, short hairpin RNA, small interfering RNA, and the like. In yet another embodiment, the present compositions can include vitamin C; nutritional supplements including vitamins, CoQ10, flavonoids, free fatty acid, alpha lipoic acid, acai, gogi, mango, pomergrante, L-carnitine, selenium; etc.

Accordingly, treatment systems, including immunotherapy treatment systems are contemplated that include an immunotherapy and an immunoresponse regulator composition. Such a treatment system can be delivered to a subject separately or mixed together in a single delivery composition or multiple delivery compositions. In some examples, the immunotherapy component is delivered first, followed by the delivery of the immunoresponse regulator at any various times following the immunotherapy, which may vary from therapy to therapy. For example, the immunoresponse regulator administration can be performed, minutes, hours, days, or in some case weeks after the administration of the immunotherapy component. In other examples, the immunoresponse regulator is delivered first, followed by the delivery of the immunotherapy component at any various times following the immunoresponse regulator, which may vary from therapy to therapy. For example, the immunotherapy component administration can be performed, minutes, hours, days, or in some case weeks after the administration of the immunoresponse regulator.

For example, an immunotherapy treatment system can include an immunoresponse regulator composition and an adoptive cellular therapy component, such as Chimeric Antigen Receptor (CAR) T Cell therapy, Chimeric Antigen Receptor (CAR) natural killer (NK) cell therapy, tumor infiltrating lymphocyte (TIL) therapy, endogenous T cell (ETC) therapy, for example.

In another example, treatment systems, including immunotherapy treatment systems are contemplated that include various cancer vaccines and an immunoresponse regulator composition. Such a treatment system can be delivered to a subject separately or mixed together in a single delivery composition or multiple delivery compositions.

In some examples, the immunotherapy component is delivered first, followed by the delivery of the immunoresponse regulator at various times following the immunotherapy, which may vary from therapy to therapy. For example, the immunoresponse regulator administration can be performed, minutes, hours, days, or in some case weeks after the administration of the cancer vaccine component. In other examples, the immunoresponse regulator is delivered first, followed by the delivery of the cancer vaccine component at various times following the immunoresponse regulator, which may vary from therapy to therapy. For example, the cancer vaccine component administration can be performed, minutes, hours, days, or in some case weeks after the administration of the immunoresponse regulator. The immunoresponse regulator can include lactate, iodoacetate, pyruvate, and various halopyruvates, including salts and acids thereof. On specific example an immunoresponse regulator includes 3-BP.

In another example, treatment systems, including immunotherapy treatment systems are contemplated that include a cytokine therapy component and an immunoresponse regulator composition. Such a treatment system can be delivered to a subject separately or mixed together in a single delivery composition or multiple delivery compositions. In some examples, the cytokine therapy component is delivered first, followed by the delivery of the immunoresponse regulator at various times following the cytokine therapy component, which may vary from therapy to therapy. For example, the immunoresponse regulator administration can be performed, minutes, hours, days, or in some case weeks after the administration of the cytokine therapy component. In other examples, the immunoresponse regulator is delivered first, followed by the delivery of the cytokine therapy component at various times following the immunoresponse regulator, which may vary from therapy to therapy. For example, the cytokine therapy component administration can be performed, minutes, hours, days, or in some case weeks after the administration of the immunoresponse regulator. The immunoresponse regulator can include lactate, iodoacetate, pyruvate, and various halopyruvates, including salts and acids thereof. On specific example an immunoresponse regulator includes 3-BP.

In another example, treatment systems, including immunotherapy treatment systems are contemplated that include a monoclonal antibody (mAb) component and an immunoresponse regulator composition. Such a treatment system can be delivered to a subject separately or mixed together in a single delivery composition or multiple delivery compositions. In some examples, the mAb component is delivered first, followed by the delivery of the immunoresponse regulator at various times following the mAb component, which may vary from therapy to therapy. For example, the immunoresponse regulator administration can be performed, minutes, hours, days, or in some case weeks after the administration of the mAb component. In other examples, the immunoresponse regulator is delivered first, followed by the delivery of the mAb component at various times following the immunoresponse regulator, which may vary from therapy to therapy. For example, the mAb component administration can be performed, minutes, hours, days, or in some case weeks after the administration of the immunoresponse regulator. The mAb component can include, without limitation, mAb compositions such as Alemtuzumab, Bevacizumab, Cetuximab, Dostarlimab, Gemtuzumab ozogamicin, Ipilimumab, Nivolumab, Ofatumumab, Panitumumab, Pembrolizumab, Ranibizumab, Rituximab, and Trastuzumab. Furthermore, an mAb component can be a conjugated mAb and/or a bispecific mAb therapy. The immunoresponse regulator can include lactate, iodoacetate, pyruvate, and various halopyruvates, including salts and acids thereof. On specific example an immunoresponse regulator includes 3-BP.

In another example, treatment systems, including immunotherapy treatment systems, are contemplated that include an immune checkpoint regulator component and an immunoresponse regulator composition. Such a treatment system can be delivered to a subject separately or mixed together in a single delivery composition or multiple delivery compositions. In some examples, the immune checkpoint regulator component is delivered first, followed by the delivery of the immunoresponse regulator at various times following the immune checkpoint regulator component, which may vary from therapy to therapy. For example, the immunoresponse regulator administration can be performed, minutes, hours, days, or in some case weeks after the administration of the immune checkpoint regulator component. In other examples, the immunoresponse regulator is delivered first, followed by the delivery of the immune checkpoint regulator component at various times following the immunoresponse regulator, which may vary from therapy to therapy. For example, the immune checkpoint regulator component administration can be performed, minutes, hours, days, or in some case weeks after the administration of the immunoresponse regulator.

Non-limiting examples of immune checkpoint regulator components include PD-1 and PD-L1 inhibitors, CTLA-4 inhibitors, LAG-3 inhibitors, and the like. Specific immune checkpoint regulator components that target PD-1 include Pembrolizumab (Keytruda), Nivolumab (Opdivo), and Cemiplimab (Libtayo), and examples that target PD-L1 include Atezolizumab (Tecentriq), Avelumab (Bavencio), and Durvalumab (Imfinzi). Additionally, examples of immune checkpoint regulator components that target CTLA-4 and LAG-3 inhibitors include Ipilimumab (Yervoy) and Relatlimab, respectively. The immunoresponse regulator can include lactate, iodoacetate, pyruvate, and various halopyruvates, including salts and acids thereof. On specific example an immunoresponse regulator includes 3-BP.

1. A system for the treatment of a condition susceptible to overactivation of a subject's immune system, comprising:
an immunotherapeutic capable of treating a condition by modulating activity of a subject's immune system;
an immunoresponse regulator composition, including;
3-bromopyruvic acid (3-BP) and salts thereof;
at least one sugar to stabilize the 3-BP by substantially preventing the 3-BP from hydrolyzing; and
a biological buffer present in an amount sufficient to at least partially deacidify and neutralize metabolic by-products of the 3-BP.

2. The system of claim 1, wherein the immunotherapeutic is capable of treating an immunodeficiency, a hypersensitivity reaction, an autoimmune disease, pathogenic infections, a tissue or organ transplantation, a cancer, an inflammatory disorder, an infectious disease, immunization reactions, or a combination thereof.

3. The system of claim 2, wherein the immunotherapeutic is capable of treating a cancer.

4. The system of claim 1, wherein the at least one sugar is a member selected from the group consisting of gluconic acid, glucuronic acid, mannitol, erythritol, isomalt, lactitol, maltitol, sorbitol, xylitol, dulcitol, ribitol, inositol, glycerol, ethylene glycol, threitol, arabitol, galactitol, fucitol, iditol, volemitol, maltotriitol, maltotetraitol, polyglycitol, and a combination thereof.

5. The system of claim 1, wherein the immunoresponse regulator composition further comprises a second sugar selected from the group consisting of mannitol, erythritol, isomalt, lactitol, maltitol, sorbitol, xylitol, dulcitol, ribitol, inositol, sorbitol, and combinations thereof.

6. The system of claim 1, wherein the immunoresponse regulator composition further comprises a second sugar and a third sugar independently selected from mannitol, erytritol, isomalt, lactitol, maltitol, sorbitol, xyolitol, dulcitol, ribitol, inositol, sorbitol, or a combination thereof.

7. The system of claim 1, wherein the immunoresponse regulator composition further comprises at least one sugar selected from glycerol, inositol, and sorbitol.

8. The system of claim 1, wherein the immunoresponse regulator composition further comprises d-lactic acid and epinephrine.

9. The system of claim 1, wherein the immunoresponse regulator composition further comprises a glycolysis inhibitor.

10. The system of claim 9, wherein the glycolysis inhibitor is 2-deoxyglucose.

11. The system of claim 10, wherein the 2-deoxyglucose is in a concentration from about 1 mM to about 5 mM.

12. The system of claim 1, wherein the biological buffer is selected from a citrate buffer, a phosphate buffer, and an acetate buffer.

13. The system of claim 1, wherein the biological buffer is a citrate buffer.

14. The system of claim 1, wherein the immunoresponse regulator composition further comprises at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogenesis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, gogi, mango, pomergrante, L-carnitine, selenium; a less biologically active amino acid as compared to its isomer; and mixtures thereof.

15. The system of claim 1, wherein the immunoresponse regulator composition further comprises a hexokinase inhibitor.

16. A method for treating a condition that is susceptible to overactivation of a subject's immune system, comprising:
administering to a subject an immunotherapeutic capable of increasing the activity of the immune system to treat the condition;
delivering an immunoresponse regulator composition to the subject to eliminate or otherwise pacify hyperactivated immune cells, the immunoresponse regulator including;
3-bromopyruvic acid (3-BP) and salts thereof;
at least one sugar to stabilize the 3-BP by substantially preventing the 3-BP from hydrolyzing; and
a biological buffer present in an amount sufficient to at least partially deacidify and neutralize metabolic by-products of the 3-BP.

17. The system of claim 16, wherein the condition is an immunodeficiency, a hypersensitivity reaction, an autoimmune disease, pathogenic infections, a tissue or organ transplantation, a cancer, an inflammatory disorder, an infectious disease, immunization reactions, or a combination thereof.

18. The method of claim 17, wherein the condition is a cancer.

19. The method of claim 16, further comprising determining overactivation of the subject's immune system prior to administering the immunoresponse regulator.

20. The method of claim 16, where overactivation of the subject's immune system further comprises production of detrimental amounts of cytokines.

21. The method of claim 20, further comprising administering the immunoresponse regulator to reduce the detrimental amounts of cytokines.

22. The method of claim 21, wherein delivery of the immunoresponse regulator composition inhibits cellular energy production in hyperactivated immune cells, thus limiting the ability of the hyperactivated immune cells to generate further cytokines.


SEQUENCE LISTING (10 sequences)

SEQ ID NO: 1 - protein, 917 aa, Homo sapiens
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 2 - protein, 916 aa, Homo sapiens
MDCEHSLSLPCRGAEAWEIGIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 3 - protein, 921 aa, Homo sapiens
MGQICQRESATAAEKPKLHLLAESEIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 4 - protein, 905 aa, Homo sapiens
MAKRALRDFIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 5 - protein, 917 aa, Homo sapiens
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 6 - protein, 25 aa, Homo sapiens
MIAAQLLAYYFTELKDDQVKKIDKY

SEQ ID NO: 7 - protein, 25 aa, Homo sapiens
MDCEHSLSLPCRGAEAWEIGIDKYL

SEQ ID NO: 8 - protein, 25 aa, Homo sapiens
MGQICQRESATAAEKPKLHLLAESE

SEQ ID NO: 9 - protein, 25 aa, Homo sapiens
MAKRALRDFIDKYLYAMRLSDETLI

SEQ ID NO: 10 - protein, 25 aa, Homo sapiens
MIASHLLAYFFTELNHDQVQKVDQY